(19) United States
(12) Patent Application Publication
(10) Pub. No.: US 2016/0235425 A1
(43) Pub. Date: Aug. 18, 2016

(54) Surgical Instrument

(71) Applicant: Japanese Organization for Medical Device Development, Inc., Tokyo (JP)

(72) Inventor: TAKAO OHKI, Tokyo (JP)

(21) Appl. No.: 14/622,926

(22) Filed: Feb. 16, 2015

(51) Int. Cl. A61B 17/285; A61B 17/28

(52) U.S. Cl. CPC A61B 17/285; A61B 17/282; A61B 17/2816

(57) ABSTRACT

A surgical instrument which comprises a first and a second working sections 11a, 11b. The working sections comprises a first and a second gripping surface 15a, 15b at the end portions. The first and the second gripping surface 15a, 15b comprise flat peripheral areas 19a, 19b and a plurality of protrusions 21a, 23a, 12b, 23b inside the flat peripheral areas 19a, 19b.

### TECHNICAL FIELD

The present invention relates to a surgical instrument with various functions.

### BACKGROUND OF THE ART

WO2014/103096 discloses a surgical instrument.

### OBJECT

An object of the invention is to provide a surgical instrument with various functions.

Especially, the invention is to provide a surgical instrument, such as a forceps, that has enough grip power and can grip surgical string.

Another object of the invention is to provide a surgical instrument that can grip surgical strings and living tissues without damaging them.

### MEANS FOR SOLVING THE OBJECT

Any of the above objects are solved by the invention claimed.

The first aspect of the invention relates to a surgical instrument which comprises a first working sections 11a and a second working section 11b. The first and the second working sections 11a, 11b are pivotally connected such that they can be opened or closed. Because the example of the surgical instrument is a forceps, a skilled person would understand the features of the invention.

The first working section 11a comprises a first cutting section 13a and a first gripping section 17a. The first gripping section 17a comprises a first gripping surface 15a.

The second working section 11b comprises a second cutting section 13b and a second gripping section 17b. The second gripping section 17b comprises a second gripping surface 15b.

The first cutting section 13a and the second cutting section 13b are configured to cut a first object.

The first gripping surface 15a comprises a first flat peripheral area 19a and a first plurality of protrusions 21a, 23a inside the first flat peripheral area 19a.

The second gripping surface 15b comprises a second flat peripheral area 19b and a second plurality of protrusions 21b, 23b inside the second flat peripheral area 19b.

The first gripping surface 15a and the second gripping surface 15b are configured to be at the end portion of the first and the second working sections 11a, 11b, respectively. The first gripping surface 15a and the second gripping surface 15b are configured to grip a second object when the first and the second working sections 11a, 11b are closed.

A preferred embodiment of the invention is that the first and the second working sections 11a, 11b further comprise a first and a second detaching section 20a, 20b, respectively. The first and the second detaching section 20a, 20b are configured to detaching living tissue when the first and the second working sections 11a, 11b are opening.

A preferred embodiment of the invention is that the first plurality of protrusions 21a, 23a and the second plurality of protrusions 21b, 23b are configured to grip living tissue.

A preferred embodiment of the invention is that the width of the first flat peripheral area 19a and the second flat peripheral area 19b is 0.1 mm to 3 mm. A preferred embodiment of the invention is that each of the first plurality of protrusions 21a, 23a and the second plurality of protrusions 21b, 23b has the width of 0.1 mm to 0.6 mm.

A preferred embodiment of the invention is that the first plurality of protrusions 21a, 23a have three line-shaped grooves 25a, 27a, 29a arranged in parallel or the second plurality of protrusions 21b, 23b have three line-shaped grooves 25b, 27b, 29b arranged in parallel.

A preferred embodiment of the invention is that the surgical instrument is a forceps.

### TECHNICAL EFFECT

Because the surgical instrument has flat peripheral areas and a plurality of protrusions inside the flat peripheral areas, it can grip surgical strings and living tissues without damaging them.

### BRIEF EXPLANATION OF FIGURES

FIGS. 1a and 1b show a surgical instrument of the invention.

FIG. 2 shows an enlarged part of FIG. 1.

FIGS. 3a, 3b, 3c, 3d show an edge portion of the surgical instrument.

FIG. 4 depicts the portion of the first gripping section and the second gripping section.

FIG. 5 depicts variation of gripping section.

FIG. 6 depicts the design figure to manufacture a forceps.

FIG. 7A depicts the front view of the manufactured forceps with length of 183 mm.

FIG. 7B depicts the side view of the forceps of FIG. 7A.

FIG. 7C depicts the enlarged image of the end section of the forceps depicted in

FIG. 7A which includes the gripping sections of the manufactured forceps.

FIG. 8A depicts the front view of the manufactured forceps with length of 235 mm.

FIG. 8B depicts the side view of the forceps of FIG. 8A.

FIG. 9 is a picture of the gripping sections of the manufactured forceps.

### BEST MODE FOR CARRYING OUT THE INVENTION

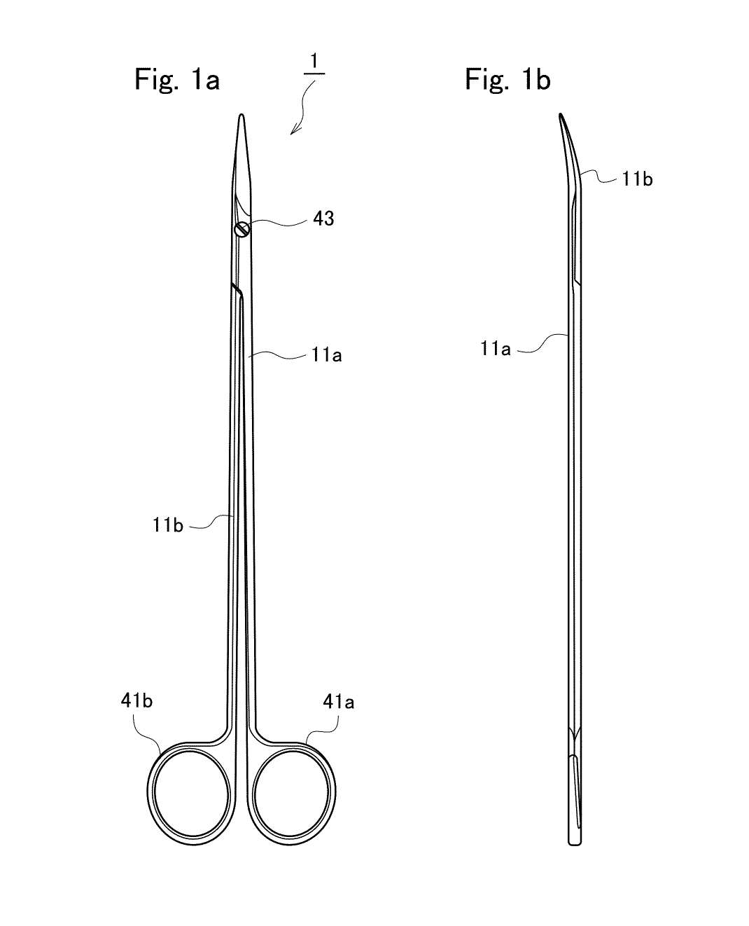

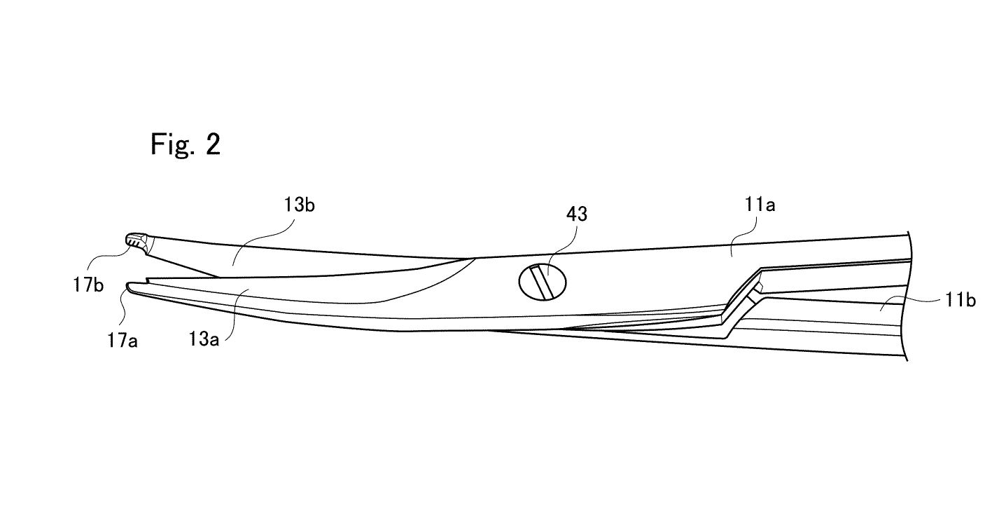

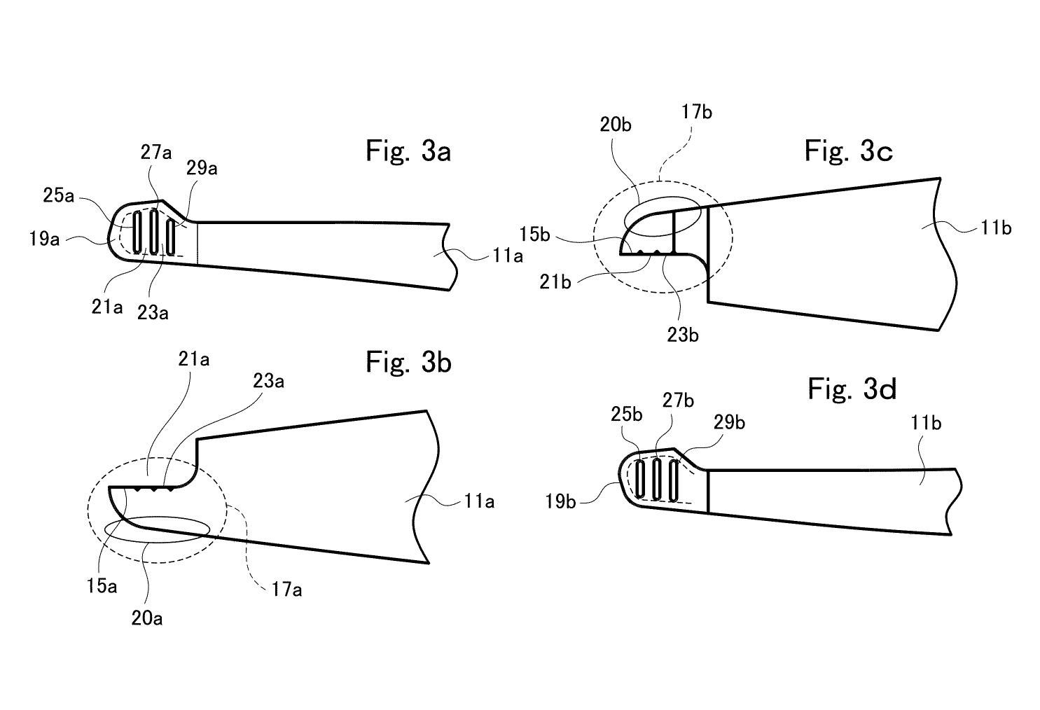

The invention is explained with reference of figures. FIGS. 1a and 1b show a surgical instrument of the invention. FIG. 2 shows an enlarged part of FIG. 1. FIGS. 3a, 3b, 3c, 3d show an edge portion of the surgical instrument.

The first aspect of the invention relates to a surgical instrument 1 which comprises a first working sections 11a and a second working section 11b. The first and the second working sections 11a, 11b may be two blades with handles 41a, 41b. The first and the second working sections 11a, 11b are pivotally connected through a transverse pin 43 such that they can be opened or closed. Because the example of the surgical instrument is a forceps, a skilled person would understand the features of the invention and how it is used. FIG. 1b shows a side view of the surgical instrument. As shown in the figure, the surgical instrument may be curved.

The first working section 11a comprises a first cutting section 13a and a first gripping section 17a. The length of the first gripping section 17a may be from 1 mm to 5 mm, preferably from 1 mm to 4 mm, more preferably from 1.5 mm to 3 mm, and the length may be from 1.5 mm to 2.5 mm. The first gripping section 17a comprises a first gripping surface 15a. The second working section 11b comprises a second cutting section 13b and a second gripping section 17b. The second gripping section 17b comprises a second gripping surface 15b. The length of the second gripping section 17b may be from 1 mm to 5 mm, preferably from 1 mm to 4 mm, more preferably from 1.5 mm to 3 mm, and the length may be from 1.5 mm to 2.5 mm.

The first cutting section 13a and the second cutting section 13b are configured to cut a first object. The first cutting section 13a and the second cutting section 13b may have blades and both blades are configured to slide each other when a user closes the first and the second working sections 11a, 11b by means of handles 41a, 41b. When an object is put between the first cutting section 13a and the second cutting section 13b, the object is cut by means of the blades. Examples of the object are surgical string and tissues, which may be patients'.

As shown in the FIGS. 3a and 3b, the first gripping surface 15a comprises a first flat peripheral area 19a and a first plurality of protrusions 21a, 23a inside the first flat peripheral area 19a. The length of the first gripping surface 15a may be 1 mm to 3 mm, preferably it is 1.5 mm to 2.5 mm. As shown in FIG. 3b, the first gripping surface 15a is at the thin portion of the first working section 11a, which is at the end portion of the first working section 11a.

The first flat peripheral area 19a surrounds the part in which the first plurality of protrusions 21a, 23a exist and is made to be flat. A preferred embodiment of the invention is that the width of the first flat peripheral area 19a and the second flat peripheral area 19b is 0.1 mm to 3 mm. The width of the first flat peripheral area may be calculated as the distance from the edge portion of the protrusions 21a, 23a or line-shaped grooves 25a, 27a, 29a to the edge of the first gripping surface 15a.

The second gripping surface 15b is similar to the first gripping surface and it comprises a second flat peripheral area 19b and a second plurality of protrusions 21b, 23b. The second plurality of protrusions 21b, 23b are inside the second flat peripheral area 19b.

The first gripping surface 15a and the second gripping surface 15b are configured to be at the end portion of the first and the second working sections 11a, 11b, respectively. The first gripping surface 15a and the second gripping surface 15b are configured to grip a second object when the first and the second working sections 11a, 11b are closed. The second object may be surgical string or tissues.

A preferred embodiment of the invention is that the first and the second working sections 11a, 11b further comprise a first and a second detaching section 20a, 20b, respectively. The first and the second detaching section 20a, 20b are configured to detaching living tissue when the first and the second working sections 11a, 11b are opening.

A preferred embodiment of the invention is that the first plurality of protrusions 21a, 23a and the second plurality of protrusions 21b, 23b are configured to grip living tissue. Further it is preferred that the first plurality of protrusions 21a, 23a and the second plurality of protrusions 21b, 23b are configured to grip surgical string. The height of the protrusions 21a, 23a, 21b, 23b is, e.g., 0.05 to 0.1 mm. Preferred example of the height is 0.06 to 0.08 mm. It is then possible to grip surgical string without adding damage to it.

A preferred embodiment of the invention is that each of the first plurality of protrusions 21a, 23a and the second plurality of protrusions 21b, 23b has the width of 0.1 mm to 0.6 mm. Preferred example of the width is 0.2 to 0.5 mm. The width may be calculated from the top portion of one protrusion to the top portion of the neighbouring protrusion.

As shown in FIGS. 3a, 3d, a preferred embodiment of the invention is that the first plurality of protrusions 21a, 23a have three line-shaped grooves 25a, 27a, 29a arranged in parallel. Other preferred embodiment of the invention is that the second plurality of protrusions 21b, 23b have three line-shaped grooves 25b, 27b, 29b arranged in parallel. The depth of grooves is 0.05 to 0.1 mm. Preferred example of the depth is 0.06 to 0.08 mm.

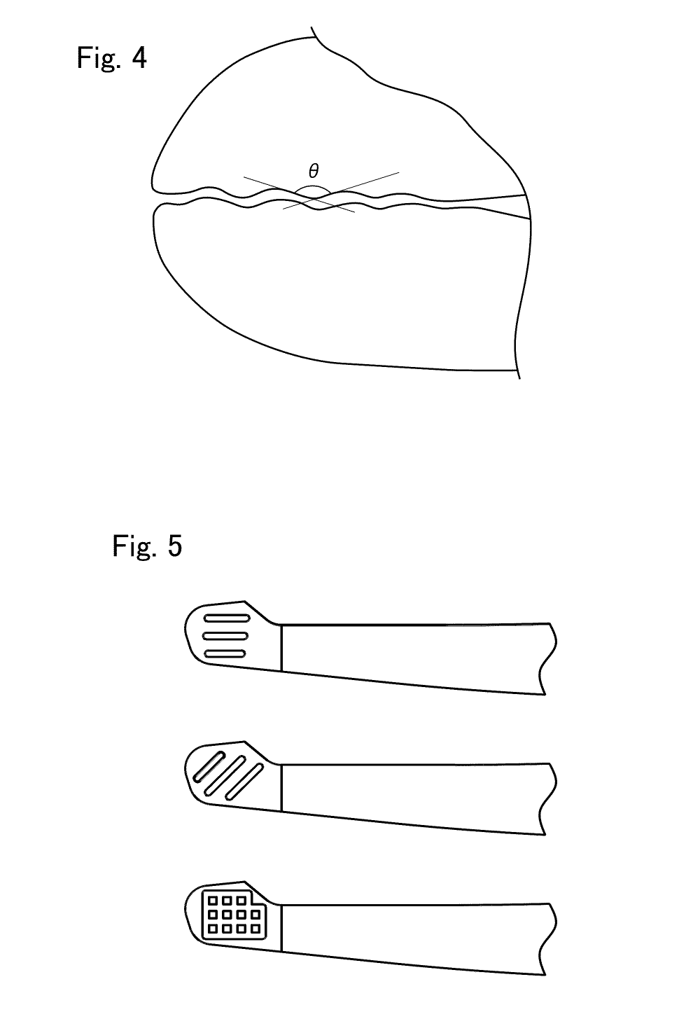

FIG. 4 depicts the portion of the first gripping section and the second gripping section. It is preferred that the top angle of protrusion is from 100 to 150 degrees and preferably from 120 to 140 degrees. The top angle is depicted in the FIG. 4.

FIG. 5 depicts variation of gripping section. The direction of grooves and protrusions may be in a direction of the first and the second working sections 11a, 11b. The direction may be in a tilt direction. The protrusions may be at lattice points.

A preferred embodiment of the invention is that the surgical instrument is a forceps.

Because of the above explained feature, even though the first and second gripping sections 17a, 17b are small the surgical instrument has high grip strength and sufficient to grip objects.

How to use the surgical instrument 1 is explained. A user inserts fingers into handles 41a, 41b. When the user opens the handles 41a, 41b, the first working sections 11a and the second working section 11b pivotally slide to open them.

When the user tries to detach living tissue, the user inserts the edge portion of the surgical instrument 1 and moves it with opening the first working sections 11a and the second working section 11b. When the user ties to grip living tissue or surgical elements, such as surgical string, the user puts the object between the protrusions or the flat peripheral areas. Then the user makes the first working sections 11a and the second working section 11b pivotally and slightly be closed to grip the object.

### Working Example 1

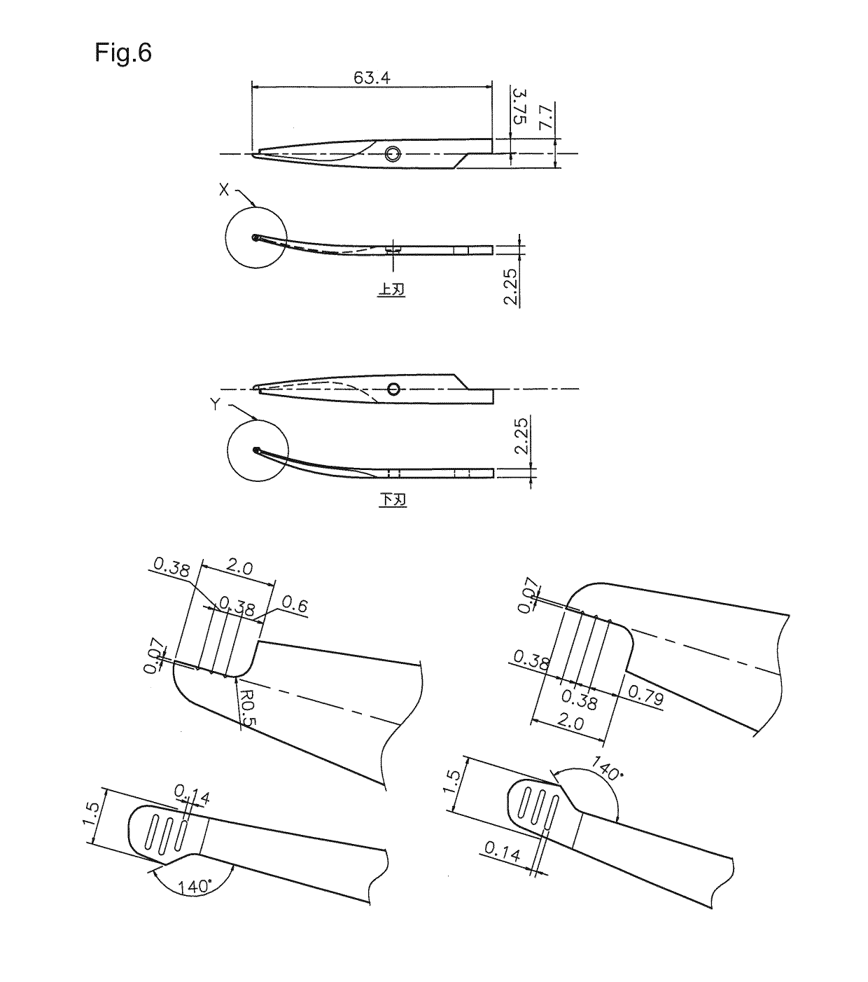

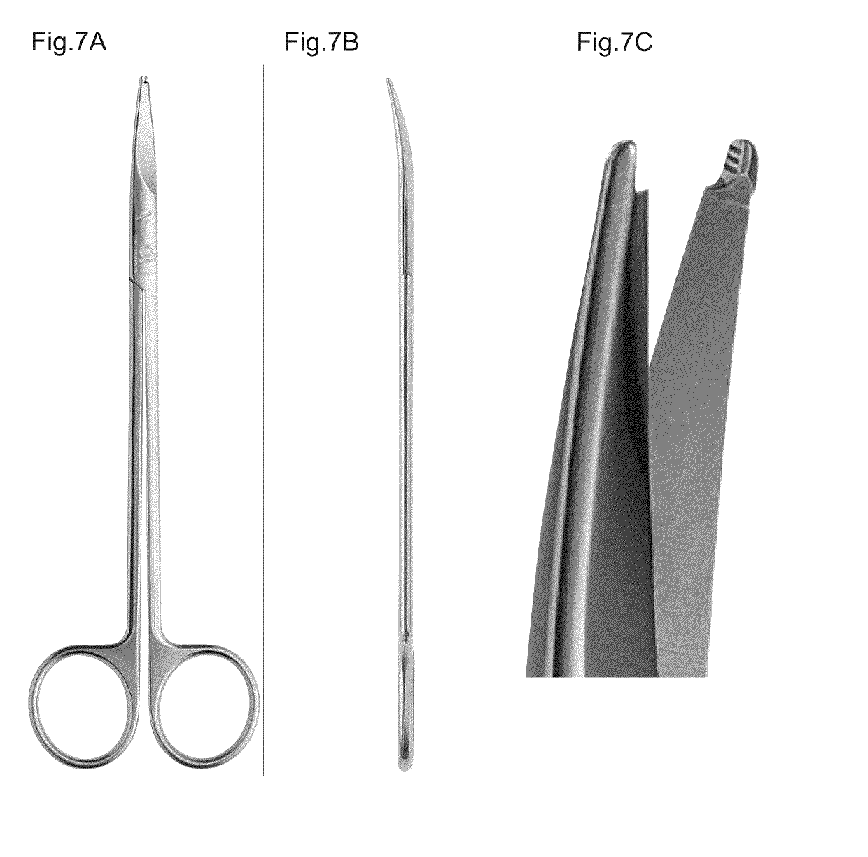

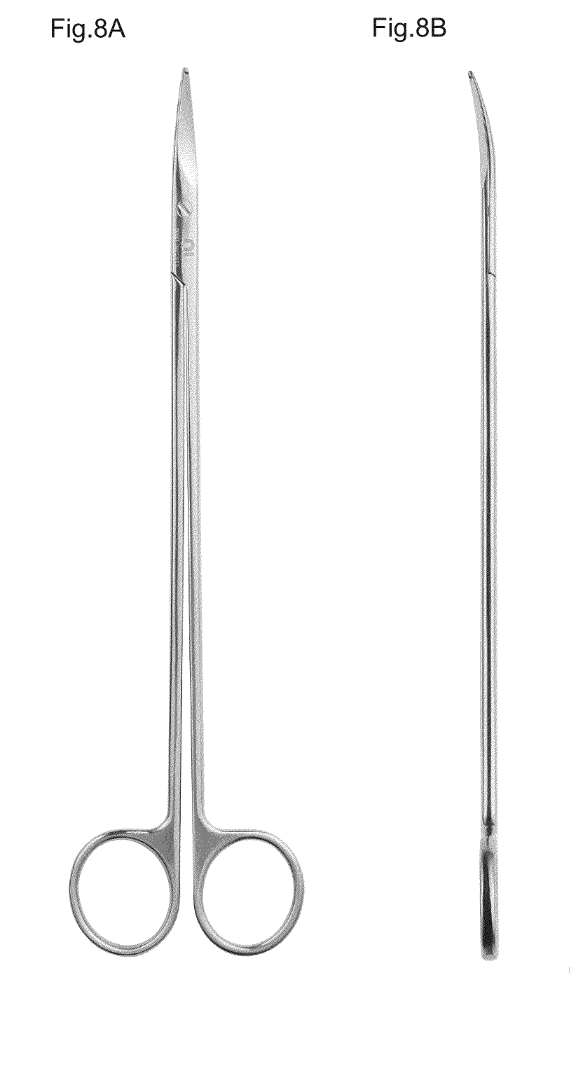

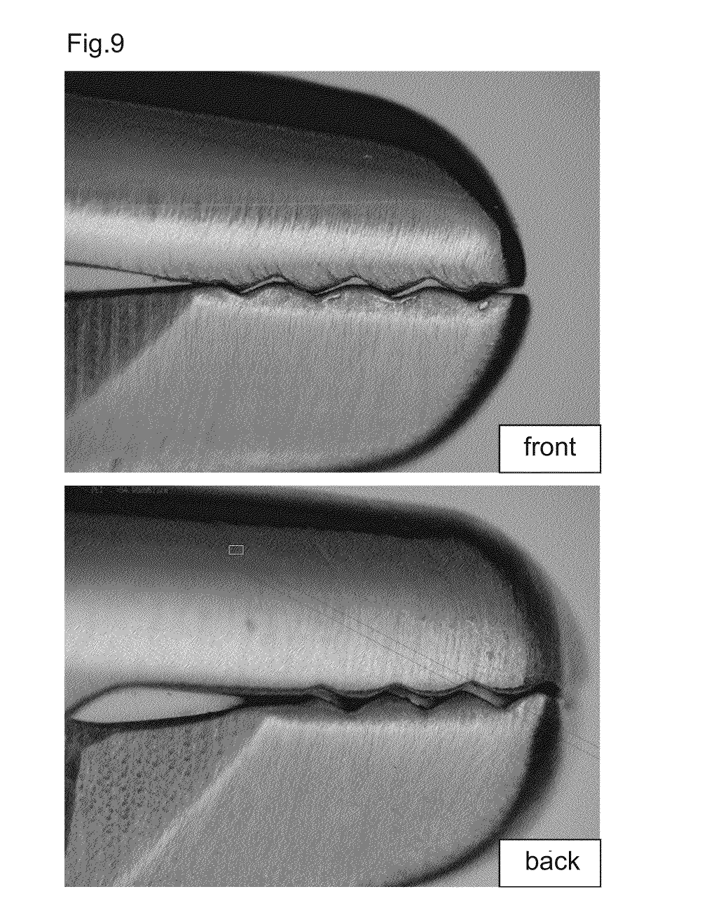

FIG. 6 depicts the design figure to manufacture a forceps. Based on the design figure, forceps are manufactured using stainless steel. FIGS. 7A to 7C, 8A and 8B are pictures of the manufactured forceps. FIG. 7A depicts the front view of the manufactured forceps with length of 183 mm. FIG. 7B depicts the side view of the forceps. FIG. 7C depicts the enlarged image of the end section of the forceps depicted in FIG. 7A which includes the gripping sections of the manufactured forceps. FIG. 8A depicts the front view of the manufactured forceps with length of 235 mm. FIG. 8B depicts the side view of the forceps. FIG. 9 is a picture of the gripping sections of the manufactured forceps.

1. A surgical instrument which comprises a first and a second working sections which are connected so as to be capable of opening and closing,
the first working section comprises a first cutting section and a gripping section, the gripping section comprising a first gripping surface,
the second working section comprises a second cutting section and a gripping section, the gripping section comprising a second gripping surface,
wherein the first cutting section and the second cutting section are configured to cut a first object,
wherein the first gripping surface comprises a first flat peripheral area and a first plurality of protrusions inside the first flat peripheral area,
wherein the second gripping surface comprises a second flat peripheral area and a second plurality of protrusions inside the second flat peripheral area,
wherein the first gripping surface and the second gripping surface are configured to be at the end portion of the first and the second working sections, respectively, and grip a second object when the first and the second working sections are closed.

2. The surgical instrument in accordance with claim 1,
wherein the first and the second working sections further comprise a first and a second detaching section, respectively, the first and the second detaching section being configured to detaching living tissue when the first and the second working sections are opening.

3. The surgical instrument in accordance with claim 1,
wherein the first plurality of protrusions and the second plurality of protrusions are configured to grip living tissue.

4. The surgical instrument in accordance with claim 1,
wherein a width of the first flat peripheral area and the second flat peripheral area is 0.1 mm to 3 mm, and
wherein each of the first plurality of protrusions and the second plurality of protrusions has a width of 0.1 mm to 0.6 mm.

5. The surgical instrument in accordance with claim 1,
wherein the first plurality of protrusions have three line-shaped grooves arranged in parallel and the second plurality of protrusions have three line-shaped grooves arranged in parallel.

6. The surgical instrument in accordance with claim 1, wherein the surgical instrument is a forceps.
